(19) United States
(12) Patent Application Publication
(10) Pub. No.: US 2021/0389766 A1
(43) Pub. Date: Dec. 16, 2021

(54) Methods and Apparatuses for Water Body Pollution Intelligent Investigation Utilizing Unmanned Ships

(71) Applicant: CHINESE RESEARCH ACADEMY OF ENVIRONMENTAL SCIENCES, Beijing (CN)

(72) Inventors: LIEYU ZHANG, Beijing (CN); MENGYU YANG, Beijing (CN); CAOLE LI, Beijing (CN); JIAQIAN LI, Beijing (CN); CHEN ZHAO, Beijing (CN); WEI LI, Beijing (CN); XIAOGUANG LI, Beijing (CN); GUOWEN LI, BEIJING (CN)

(21) Appl. No.: 17/240,016

(22) Filed: Apr. 26, 2021

(30) Foreign Application Priority Data
Jun. 12, 2020 (CN) 202010537764X

(51) Int. Cl. G05D 1/02; G01N 33/18; G06N 20/00; B63B 79/10; B63B 79/40

(52) U.S. Cl. CPC G05D 1/0206; G01N 33/1886; B63B 79/40; B63B 79/10; G06N 20/00

(57) ABSTRACT

Embodiments of the present disclosure relate to a water body pollution intelligent investigation method and device based on unmanned ships. The method includes: determining a first pollutant concentration value of a monitored water area according to water quality data of the monitored water area; controlling an unmanned ship to cruise in the monitored water area according to a preset cruise trajectory and perform water quality collection to obtain a second pollutant concentration value of the monitored water area; determining, when there is an abnormal cruise coordinate point, a target cruise trajectory according to the second pollutant concentration value of the abnormal cruise coordinate point; and controlling the unmanned ship to cruise according to the target cruise trajectory to determine a pollution source of the monitored water area.

### CROSS REFERENCE TO RELATED APPLICATION

This application claims the benefit of Chinese Patent Application No. 202010537764X, filed Jun. 12, 2020, which is hereby incorporated by reference herein in its entirety.

### TECHNICAL FIELD

The present disclosure relates to the field of unmanned ships, in particular to a water body pollution intelligent investigation method and device based on unmanned ships.

### BACKGROUND

Aiming at the problem of water pollution, the water quality environment monitoring methods currently used in China are mainly divided into three types: a laboratory monitoring method, a mobile monitoring method and an automatic monitoring station testing method.

In the laboratory monitoring method, the staffs arrive at a sampling point to take samples by renting a ship, and perform detailed water quality analysis on the collected water samples in the laboratory and generate reports. The laboratory monitoring method is mainly used for periodic monitoring and evaluation of water quality, and the accuracy of the monitoring results of this method is generally high. The water quality testing method based on establishment of water quality testing stations is currently the main water quality testing method. This method can well resist the interference of the external environment and improve the monitoring accuracy of water quality data. The mobile monitoring method is specially designed for emergent and periodic water quality inspections. There are two main manners: one is that the tester uses a mobile monitoring ship to perform sample collection and analysis on the water quality of a point to be tested, and the other is that an unmanned facility specially equipped with a sensor for monitoring water quality is manually controlled to collect and analyze the water quality of a water area to be tested.

Among these existing water quality monitoring methods, the laboratory monitoring method is time-consuming and laborious in the implementation process, has the defects of high cost and poor real-time performance, and often fails to provide timely warning for unexpected pollution accidents, resulting in unpredictable losses. The work environment and life safety of the testers are not guaranteed, and the test data cannot be managed in an informatized manner. The establishment of water quality testing stations requires the establishment of relevant monitoring sites at various sampling points, so the investment and maintenance costs of water quality monitoring are high, and a certain destructive impact will be caused on the environment of nearby water areas. Moreover, a wide range of water areas must be tested, so more capital cost needs to be invested for increases of the construction scale and number of sites. The existing mobile monitoring method requires the operator to manipulate a mobile monitoring ship or unmanned facility to perform water quality collection and analysis, which is a waste of manpower.

In terms of the above problems, no effective solutions have been proposed yet.

### SUMMARY

Embodiments of the present disclosure provides a water body pollution intelligent investigation method and device based on unmanned ships to at least solve the technical problem of the waste of manpower caused by the fact that a mobile monitoring method in the related art needs to manually manipulate a mobile unmanned ship to perform water quality collection and analysis.

According to one aspect of the embodiments of the present disclosure, there is provided a water body pollution intelligent investigation method based on unmanned ships, including: determining a first pollutant concentration value of a monitored water area according to water quality data of the monitored water area; controlling an unmanned ship to cruise in the monitored water area according to a preset cruise trajectory and perform water quality collection to obtain a second pollutant concentration value of the monitored water area; determining, when there is an abnormal cruise coordinate point, a target cruise trajectory according to the second pollutant concentration value of the abnormal cruise coordinate point, where a difference between the first pollutant concentration value and the second pollutant concentration value of the abnormal cruise coordinate point is greater than a preset threshold; and controlling the unmanned ship to cruise according to the target cruise trajectory to determine a pollution source of the monitored water area.

Further, the determining the first pollutant concentration value of the monitored water area according to the water quality data of the monitored water area includes: acquiring the water quality data of the monitored water area, where the water quality data includes satellite data of the monitored water area and a plurality of sensor data; and inputting the satellite data and the plurality of sensor data into a water area pollution analysis model to obtain the first pollutant concentration value, where the water area pollution analysis model is previously trained according to the water quality data.

Further, the determining, when there is an abnormal cruise coordinate point, the target cruise trajectory according to the second pollutant concentration value of the abnormal cruise coordinate point includes: acquiring the abnormal cruise coordinate point; determining a first cruise trajectory according to a circle with a preset radius centered on the abnormal cruise coordinate point; controlling the unmanned ship to cruise according to the first cruise trajectory and collect at least two pollutant concentration values of at least two first cruise coordinate points; and determining the target cruise trajectory according to the at least two pollutant concentration values.

Further, the acquiring the abnormal cruise coordinate point includes: acquiring pollutant concentration values of a plurality of first cruise coordinate points in the preset cruise trajectory; and sorting the pollutant concentration values of the plurality of first cruise coordinate points, and determining that the first cruise coordinate point with the largest pollutant concentration value is the abnormal cruise coordinate point. Further, the acquiring the abnormal cruise coordinate point includes: acquiring pollutant concentration values corresponding to a plurality of first cruise coordinate points in the preset cruise trajectory; acquiring at least two reference coordinate points of which pollutant concentration values are greater than a preset pollutant concentration threshold among the plurality of first cruise coordinate points; and determining that a center point of the at least two reference coordinate points is the abnormal cruise coordinate point.

Further, the controlling the unmanned ship to cruise according to the target cruise trajectory to determine the pollution source of the monitored water area includes: acquiring pollutant concentration values of a plurality of second cruise coordinate points in the target cruise trajectory; determining that the second cruise coordinate point with the largest pollutant concentration value is a pollution source coordinate point of the pollution source; and controlling the unmanned ship to perform water quality collection and image collection at the pollution source coordinate point.

According to another aspect of the embodiments of the present disclosure, there is further provided a water body pollution intelligent investigation device based on unmanned ships, including: a first determining unit, configured to determine a first pollutant concentration value of a monitored water area according to water quality data of the monitored water area; a first control unit, configured to control an unmanned ship to cruise in the monitored water area according to a preset cruise trajectory and perform water quality collection to obtain a second pollutant concentration value of the monitored water area; a second determining unit, configured to determine, when there is an abnormal cruise coordinate point, a target cruise trajectory according to the second pollutant concentration value of the abnormal cruise coordinate point, where a difference between the first pollutant concentration value and the second pollutant concentration value of the abnormal cruise coordinate point is greater than a preset threshold; and a second control unit, configured to control the unmanned ship to cruise according to the target cruise trajectory to determine a pollution source of the monitored water area.

Further, the first determining unit includes: an acquisition module, configured to acquire the water quality data of the monitored water area, where the water quality data includes satellite data of the monitored water area and a plurality of sensor data; and a processing module, configured to input the satellite data and the plurality of sensor data into a water area pollution analysis model to obtain the first pollutant concentration value, where the water area pollution analysis model is previously trained according to the water quality data.

According to another aspect of the embodiments of the present disclosure, there is further provided an unmanned ship, including: a processing unit, configured to determine a first pollutant concentration value of a monitored water area according to water quality data of the monitored water area; and a control unit, configured to control the unmanned ship to cruise in the monitored water area according to a preset cruise trajectory and perform water quality collection to obtain a second pollutant concentration value of the monitored water area; where the processing unit is further configured to determine, when there is an abnormal cruise coordinate point, a target cruise trajectory according to the second pollutant concentration value of the abnormal cruise coordinate point, where a difference between the first pollutant concentration value and the second pollutant concentration value of the abnormal cruise coordinate point is greater than a preset threshold; and the control unit is further configured to control the unmanned ship to cruise according to the target cruise trajectory to determine a pollution source of the monitored water area.

According to another aspect of the embodiments of the present disclosure, there is further provided a storage medium, where the storage medium includes a stored program, where when the program is running, the water body pollution intelligent investigation method based on the unmanned ships as described above is executed.

In the embodiments of the present disclosure, the first pollutant concentration value of the monitored water area is determined according to the water quality data of the monitored water area; the unmanned ship is controlled to cruise in the monitored water area according to the preset cruise trajectory and perform the water quality collection to obtain the second pollutant concentration value of the monitored water area; when there is an abnormal cruise coordinate point, the target cruise trajectory is determined according to the second pollutant concentration value of the abnormal cruise coordinate point; and the unmanned ship is controlled to cruise according to the target cruise trajectory to determine the pollution source of the monitored water area.

The unmanned ship can automatically plan the cruise trajectory according to the actually measured pollutant concentration value to trace the pollution source of the water area, thereby solving the technical problem of the waste of manpower caused by the fact that a mobile monitoring method in the related art needs to manually manipulate a mobile unmanned ship to perform water quality collection and analysis.

### BRIEF DESCRIPTION OF THE DRAWINGS

In order to more clearly illustrate the technical solutions of embodiments of the present disclosure, the accompanying drawings that need to be used in the description of the embodiments or the prior art will be briefly described below. Obviously, the accompanying drawings in the following description are only some embodiments of the present disclosure, and those of ordinary skill in the art can obtain other accompanying drawings according to these accompanying drawings without any creative effort.

FIG. 1 is a schematic diagram of an optional unmanned ship system according to an embodiment of the present disclosure;

FIG. 2 is a schematic diagram of an optional water area monitoring system according to an embodiment of the present disclosure;

FIG. 3 is a schematic diagram of an optional water body pollution intelligent investigation method based on unmanned ships according to an embodiment of the present disclosure;

FIG. 4 is a schematic diagram of an optional first cruise trajectory according to an embodiment of the present disclosure;

FIG. 4a is a schematic diagram of another optional first cruise trajectory according to an embodiment of the present disclosure;

FIG. 5a is a schematic diagram of an optional abnormal cruise coordinate point according to an embodiment of the present disclosure;

FIG. 5b is a schematic diagram of an optional abnormal cruise coordinate point according to an embodiment of the present disclosure;

FIG. 6 is a schematic diagram of an optional water body pollution intelligent investigation device based on unmanned ships according to an embodiment of the present disclosure; and

FIG. 7 is a schematic diagram of an optional unmanned ship according to an embodiment of the present disclosure.

### DETAILED DESCRIPTION

In order to make the objectives, technical solutions and advantages of the embodiments of the present disclosure clearer, the technical solutions in the embodiments of the present disclosure will be clearly and completely described below in conjunction the accompanying drawings in the embodiments of the present disclosure. It is apparent that the described embodiments are a part of the embodiments of the present disclosure, rather than all the embodiments. All other embodiments obtained by those of ordinary skill in the art based on the embodiments in the present disclosure without creative efforts shall fall within the protection scope of the present disclosure.

It should be noted that relational terms such as “first” and “second” herein are only used to distinguish one entity or operation from another entity or operation, and do not necessarily require or imply any such actual relationship or sequence between these entities or operations.

### Embodiment 1

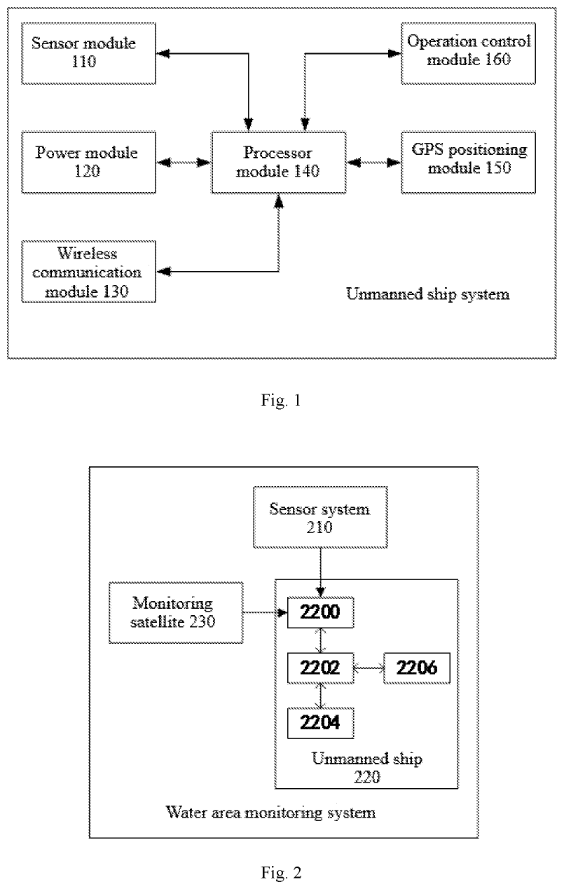

Before introducing the technical solution of the present application, the application scenario of the technical solution of this embodiment is first introduced. A water body pollution intelligent investigation method based on unmanned ships in this embodiment is mainly applied to an unmanned ship system as shown in FIG. 1. The unmanned ship includes a sensor module 110, a power module 120, a wireless communication module 130, a processor module 140, a GPS positioning module 150 and an operation control module 160. The sensor module 110 includes a water quality sensor and other sensors. The water quality sensor is configured to collect water quality data. The other sensors include, but are not limited to, a water body flow velocity sensor and an obstacle perceptron sensor. The power module 120 is configured to provide cruise power for the unmanned ship. The wireless communication module 130 is configured to perform communication and transmit data between the unmanned ship and a preset server. The processor module 140 is configured to process data, for example, to determine the concentration value of pollutants in water according to the water quality data and perform other data processing in the unmanned ship cruise process. The GPS positioning module 150 is configured to perform real-time positioning on the unmanned ship. The operation control module 160 is configured to control cruise of the unmanned ship.

In this embodiment, as shown in FIG. 2, the water area monitoring system includes a plurality of sets of sensor systems 210, unmanned ships 220 and monitoring satellites 230. In a cruise process of the unmanned ship, the wireless communication module 2200 in the unmanned ship receives a plurality of sets of sensor system test data, satellite images of the monitored water area and remote sensing data in the monitored water area, and the processor module 2202 performs computing and integration to realize edge computing, and models the integrated data by using a BP neural network to form a sensor data network, so as to predict a first pollutant concentration value of the monitored water area. Then the operation control module 2204 controls the unmanned ship to cruise in the monitored water area according to a preset cruise trajectory received by the wireless communication module 2200, the processor module 2202 controls the sensor module 2206 to perform water quality collection, and the processor module 2202 obtains an actually measured second pollutant concentration value of the monitored water area according to the water quality data collected by the sensor module 2206. In a case where the difference between the predicted first pollutant concentration value and the actually measured second pollutant concentration value of the cruise coordinate point is greater than a preset threshold, the processor module 2202 generates a target cruise trajectory according to the cruise coordinate point, and the operation control module 2204 controls the unmanned ship to cruise according to the target cruise trajectory so as to determine the pollution source of the monitored water area.

By performing water body pollution investigation based on the unmanned ships in the water area monitoring system, artificial falsification is prevented, and automatic navigation of the unmanned ship can be realized to trace the pollution source, thereby solving the technical problem of the waste of manpower caused by the fact that a mobile monitoring method in the related art needs to manually manipulate a mobile unmanned ship to perform water quality collection and analysis.

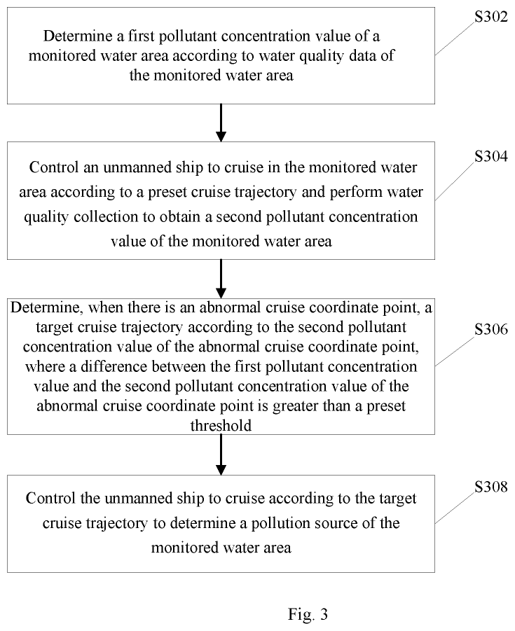

According to an embodiment of the present disclosure, there is provided a water body pollution intelligent investigation method based on unmanned ships. As shown in FIG. 3, the method includes:

S302, a first pollutant concentration value of a monitored water area is determined according to water quality data of the monitored water area.

In a specific application scenario, to predict the water quality of the monitored water area according to the water quality data, according to one solution, a BP (back propagation) neural network model is previously set at a preset server to analyze the water quality of the monitored water area based on the water quality data; and according another solution, water quality data is sent through a preset communication server to the unmanned ship, in a data processing system of which a BP neural network model is previously set, and a processor of the unmanned ship analyzes the water quality data by using the BP neural network model to predict the first pollutant concentration value of the monitored water area.

In an optional solution in this embodiment, determining the first pollutant concentration value of the monitored water area according to the water quality data of the monitored water area includes, but is not limited to: the water quality data of the monitored water area is acquired, where the water quality data includes satellite data of the monitored water area and a plurality of sensor data; and the satellite data and the plurality of sensor data are input into a water area pollution analysis model to obtain the first pollutant concentration value, where the water area pollution analysis model is previously trained according to the water quality data.

Specifically, the plurality of sets of sensor system test data, satellite photos and remote sensing data in the same flow area are sent to an unmanned ship terminal facility and are directly subjected to computing and integration to realize edge computing, the integrated data is uploaded to the processor module, and the processor module models the data by using the BP (back propagation) neural network to form a sensor data network, so as to predict the water quality data of the flow area.

S304, the unmanned ship is controlled to cruise in the monitored water area according to a preset cruise trajectory and perform water quality collection to obtain a second pollutant concentration value of the monitored water area.

In a specific application scenario, by controlling the unmanned ship to cruise in the monitored water area according to the preset cruise trajectory, in a cruise process of the unmanned ship, the unmanned ship can be controlled to perform water quality collection and testing every time the unmanned ship cruises s a preset distance, and to record the corresponding cruise coordinate points.

S306, when there is an abnormal cruise coordinate point, a target cruise trajectory is determined according to the second pollutant concentration value of the abnormal cruise coordinate point, where a difference between the first pollutant concentration value and the second pollutant concentration value of the abnormal cruise coordinate point is greater than a preset threshold.

It should be noted that the target cruise trajectory is not a definite cruise trajectory, but a general direction. The target cruise trajectory includes a cruise starting point and a cruise advancing direction. The unmanned ship starts to cruise at the cruise starting point, and automatically plans a travel route with relatively higher pollutant concentrations in the cruise advancing direction, so that the unmanned ship travels along the route.

Optionally, in this embodiment, the determining, when there is an abnormal cruise coordinate point, the target cruise trajectory according to the second pollutant concentration value of the abnormal cruise coordinate point includes, but is not limited to: the abnormal cruise coordinate point is acquired; a first cruise trajectory is determined according to a circle with a preset radius centered on the abnormal cruise coordinate point; the unmanned ship is controlled to cruise according to the first cruise trajectory and collect at least two pollutant concentration values of at least two first cruise coordinate points; and the target cruise trajectory is determined according to the at least two pollutant concentration values.

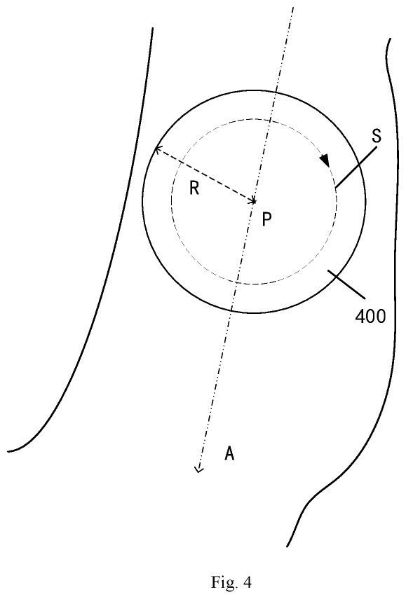

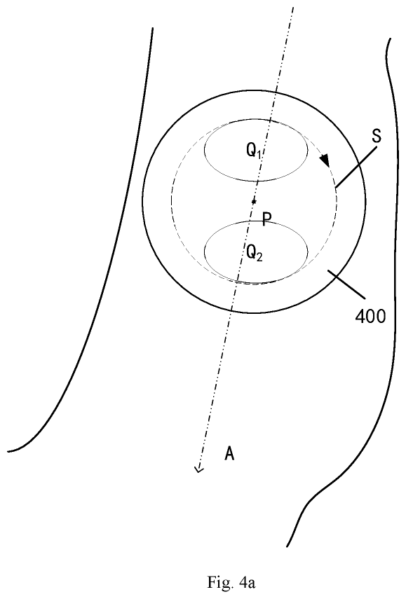

In a specific application scenario, as shown in FIG. 4, the position of an abnormal cruise coordinate point is determined, a cruise area 400 is determined by taking the abnormal cruise coordinate point P in the preset cruise trajectory A as the center of circle with a preset radius R, a first cruise trajectory S which is annular is planned in the cruise area 400, then an unmanned ship is controlled to cruise according to the first cruise trajectory, at least two pollutant concentration values of at least two first cruise coordinate points are collected in the cruise process, and then the target cruise trajectory is determined according to the at least two pollutant concentration values in the cruise area 400.

It should be noted that the cruise area is defined by taking the abnormal cruise coordinate point as the center, the first cruise trajectory is set in the cruise area, and the first cruise trajectory can be set according to actual experience and the environment of the water area, which is not limited here in this embodiment. By planning the first cruise trajectory in the cruise area, the distribution and flow direction of the pollutants are determined to trace the source of the pollutants.

In an example, according to the pollutant concentration value distribution of the cruise area 400 as shown in FIG. 4a, the pollutant concentration value in the area Q₁ is significantly higher than the pollutant concentration value in the area Q₂, and then the first cruise trajectory is planned according to the area Q₁.

In another example, in the cruise area 400 as shown in FIG. 4, the pollutant concentration values around the abnormal cruise coordinate point P are all less than the pollutant concentration value of the abnormal cruise coordinate point P, and then there may possibilities: one is that the abnormal cruise coordinate point P is the pollution source, and the other is that there is a measurement error at the abnormal cruise coordinate point P. At this time, the unmanned ship is controlled to perform water quality sampling and image collection on the environment around the abnormal cruise coordinate point.

Optionally, in this embodiment, the acquiring the abnormal cruise coordinate point includes, but is not limited to: pollutant concentration values of a plurality of first cruise coordinate points in the preset cruise trajectory are acquired; and the pollutant concentration values of the plurality of first cruise coordinate points are sorted, and the fact that the first cruise coordinate point with the largest pollutant concentration value is the abnormal cruise coordinate point is determined.

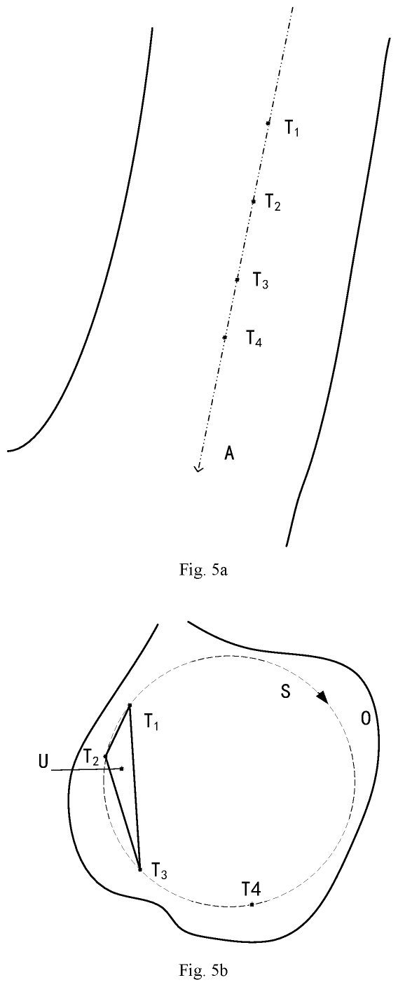

Specifically, in the cruise process of the unmanned ship along the preset cruise trajectory A, if the preset cruise trajectory is a relatively simple route, such as the linear cruise trajectory shown in FIG. 5a, then pollutant concentration values of a plurality of first cruise coordinate points T₁, T₂, T₃ and T₄ of the unmanned ship in the preset cruise process are acquired, and the pollutant concentration values of the plurality of first cruise coordinate points are sorted to obtain a sequence T₂>T₃=T₁>T₄, so that it can be concluded that the first cruise coordinate point T₂ is the abnormal cruise coordinate point.

Optionally, in this embodiment, the acquiring the abnormal cruise coordinate point includes, but is not limited to: pollutant concentration values corresponding to a plurality of first cruise coordinate points in the preset cruise trajectory are acquired; at least two reference coordinate points of which pollutant concentration values are greater than a preset pollutant concentration threshold among the plurality of first cruise coordinate points are acquired; and the fact that a center point of the at least two reference coordinate points is the abnormal cruise coordinate point is determined.

Specifically, in the cruise process of the unmanned ship along the preset cruise trajectory, if the preset cruise trajectory is a relatively simple route, such as the annular cruise trajectory S in the monitored water area O shown in FIG. 5b, then pollutant concentration values of a plurality of first cruise coordinate points T₁, T₂, T₃ and T₄ of the unmanned ship in the preset cruise process are acquired, wherein the pollutant concentration values of the first cruise coordinate points T₁, T₂ and T₃ are greater than the preset pollutant concentration threshold, and then, the abnormal cruise coordinate point is determined according to the center point U of the first cruise coordinate points.

S308, the unmanned ship to cruise is controlled according to the target cruise trajectory to determine a pollution source of the monitored water area.

Optionally, in this embodiment, the controlling the unmanned ship to cruise according to the target cruise trajectory to determine the pollution source of the monitored water area includes, but is not limited to: pollutant concentration values of a plurality of second cruise coordinate points in the target cruise trajectory are acquired; the fact that the second cruise coordinate point with the largest pollutant concentration value is a pollution source coordinate point of the pollution source is determined; and the unmanned ship is controlled to perform water quality collection and image collection at the pollution source coordinate point.

Specifically, the unmanned ship is controlled to start cruising at the cruise starting point, and automatically plan a travel route with relatively higher pollutant concentrations in the cruise advancing direction, so that the unmanned ship travels along the route, a shipborne sensor collects water quality data and water flow velocity along the travel trajectory every preset time and uploads the water quality data to a processor of the unmanned ship, and the processor analyzes and computes the pollutant concentration in the water and acquires pollutant concentration changes, thereby realizing monitoring on wide range of monitored water areas.

In this embodiment, the first pollutant concentration value of the monitored water area is determined according to the water quality data of the monitored water area; the unmanned ship is controlled to cruise in the monitored water area according to the preset cruise trajectory and perform the water quality collection to obtain the second pollutant concentration value of the monitored water area; in the case where the difference between the first pollutant concentration value and the second pollutant concentration value is greater than the preset threshold, the target cruise trajectory is determined according to the second pollutant concentration value of the cruise coordinate point; and the unmanned ship is controlled to cruise according to the target cruise trajectory to determine the pollution source of the monitored water area. The unmanned ship can automatically plan the cruise trajectory according to the actually measured pollutant concentration value to trace the pollution source of the water area, thereby solving the technical problem of the waste of manpower caused by the fact that a mobile monitoring method in the related art needs to manually manipulate a mobile unmanned ship to perform water quality collection and analysis.

It should be noted that for the foregoing method embodiments, for the sake of simple description, they are all expressed as a combination of a series of actions, but those skilled in the art should know that the present disclosure is not limited by the described sequence of actions, because according to the present disclosure, some steps can be performed in other sequence or simultaneously. Secondly, those skilled in the art should also know that the embodiments described in the specification are all preferred embodiments, and the actions and modules involved are not necessarily required by the present disclosure.

Through the description of the above implementations, those skilled in the art can clearly understand that the method according to the above embodiments can be implemented by means of software plus a necessary general hardware platform, and of course, it can also be implemented by hardware, but in many cases the former is a better implementation. Based on such an understanding, the technical solution of the present disclosure essentially or for the part that contributes to the prior art can be embodied in the form of a software product, and the computer software product is stored in a storage medium (such as ROM/RAM, magnetic disk, optical disk) and includes several instructions to enable a terminal facility (which may be a mobile phone, a computer, a server, a network facility or the like) to execute the method described in the embodiments of the present disclosure.

### Embodiment 2

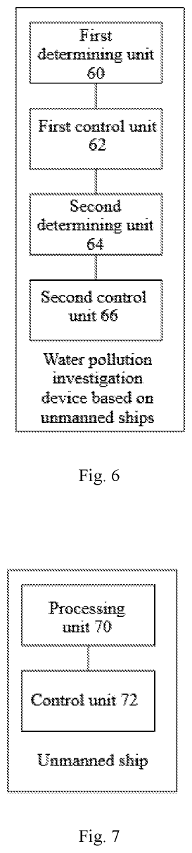

According to the embodiment of the present disclosure, there is further provided a water body pollution intelligent investigation device based on unmanned ships for implementing the above water body pollution investigation method based on unmanned ships. As shown in FIG. 6, the device includes:

1) a first determining unit 60, configured to determine a first pollutant concentration value of a monitored water area according to water quality data of the monitored water area;

2) a first control unit 62, configured to control an unmanned ship to cruise in the monitored water area according to a preset cruise trajectory and perform water quality collection to obtain a second pollutant concentration value of the monitored water area;

3) a second determining unit 64, configured to determine, when there is an abnormal cruise coordinate point, a target cruise trajectory according to the second pollutant concentration value of the abnormal cruise coordinate point, where a difference between the first pollutant concentration value and the second pollutant concentration value of the abnormal cruise coordinate point is greater than a preset threshold; and

4) a second control unit 66, configured to control the unmanned ship to cruise according to the target cruise trajectory to determine a pollution source of the monitored water area.

Optionally, in this embodiment, the first determining unit 60 includes:

1) an acquisition module, configured to acquire the water quality data of the monitored water area, where the water quality data includes satellite data of the monitored water area and a plurality of sensor data; and

2) a processing module, configured to input the satellite data and the plurality of sensor data into a water area pollution analysis model to obtain the first pollutant concentration value, where the water area pollution analysis model is previously trained according to the water quality data.

Optionally, for the specific example in this embodiment, reference may be made to the example described in Embodiment 1 above, and detailed descriptions will not be repeated here in this embodiment.

### Embodiment 3

According to an embodiment of the present disclosure, an unmanned ship for implementing the above water body pollution intelligent investigation method based on unmanned ships is further provided. As shown in FIG. 7, the unmanned ship includes:

1) a processing unit 70, configured to determine a first pollutant concentration value of a monitored water area according to water quality data of the monitored water area; and

2) a control unit 72, configured to control the unmanned ship to cruise in the monitored water area according to a preset cruise trajectory and perform water quality collection to obtain a second pollutant concentration value of the monitored water area; where

the processing unit is further configured to determine, when there is an abnormal cruise coordinate point, a target cruise trajectory according to the second pollutant concentration value of the abnormal cruise coordinate point, where a difference between the first pollutant concentration value and the second pollutant concentration value of the abnormal cruise coordinate point is greater than a preset threshold; and the control unit is further configured to control the unmanned ship to cruise according to the target cruise trajectory to determine a pollution source of the monitored water area.

Optionally, for the specific example in this embodiment, reference may be made to the example described in Embodiment 1 above, and detailed descriptions will not be repeated here in this embodiment.

### Embodiment 4

According to an embodiment of the present disclosure, there is further provided a storage medium, where the storage medium includes a stored program, where when the program is running, the water body pollution intelligent investigation method based on the unmanned ships as described above is executed.

Optionally, in this embodiment, the storage medium is configured to store program codes for executing the following steps:

S1, a first pollutant concentration value of a monitored water area is determined according to water quality data of the monitored water area;

S2, an unmanned ship is controlled to cruise in the monitored water area according to a preset cruise trajectory and perform water quality collection to obtain a second pollutant concentration value of the monitored water area;

S3, when there is an abnormal cruise coordinate point, a target cruise trajectory is determined according to the second pollutant concentration value of the abnormal cruise coordinate point, where a difference between the first pollutant concentration value and the second pollutant concentration value of the abnormal cruise coordinate point is greater than a preset threshold; and

S4, the unmanned ship is controlled to cruise according to the target cruise trajectory to determine a pollution source of the monitored water area.

Optionally, the storage medium is further configured to store the program codes for executing the steps included in the method in Embodiment 1 above, which will not be repeated here in this embodiment.

Optionally, in this embodiment, the above storage medium may include, but is not limited to a USB flash disk, a read-only memory (ROM), a random access memory (RAM), a mobile hard disk, a magnetic disk, an optical disk, or any medium that can store program codes.

Optionally, for the specific example in this embodiment, reference may be made to the example described in Embodiment 1 above, and detailed descriptions will not be repeated here in this embodiment.

The serial numbers of the embodiments of the present disclosure above are merely for the description, and do not represent the quality of the embodiments.

When the integrated unit in the embodiments above is implemented in a form of a software function unit and sold or used as an independent product, the integrated unit may be stored in the computer-readable storage medium above. Based on such an understanding, the technical solution of the disclosure essentially or for the part that contributes to the prior art or all or part of the technical solution can be embodied in the form of a software product, and the computer software product is stored in a storage medium and includes several instructions configured to enable one or more computer facilities (which may be a personal computer, a server, a network facility or the like) to execute all or part of the steps of the methods of the embodiments of the present disclosure.

In the above embodiments of the present disclosure, the description for each embodiment has its own focus. For parts that are not described in detail in a certain embodiment, reference may be made to related descriptions of other embodiments.

In the several embodiments provided in the present application, it should be understood that the disclosed client can be implemented in other ways. The device embodiments described above are only schematic. For example, the division of units is only a division of logical functions. In an actual implementation, there may be other division manners, for example, a plurality of units or components may be combined or may be integrated into another system, or some features may be omitted or not executed. In addition, the displayed or discussed mutual coupling or direct coupling or communication connection may be indirect coupling or communication connection through some interfaces, units or modules, and may be in electrical or other forms.

The units described as separate components may or may not be physically separated, and the components displayed as units may or may not be physical units, that is, they may be located in one place, or may be distributed in a plurality of network units. Part or all of the units may be selected according to actual needs to achieve the purposes of the solution of this embodiment.

In addition, the function units in each embodiment of the present disclosure may be integrated into one processing unit, or each unit may exist alone physically, or two or more units may be integrated into one unit. The above integrated unit may be implemented in the form of hardware or implemented in the form of a software function unit.

The above description is only preferred implementations of the present disclosure. It should be noted that those of ordinary skill in the art may also make several improvements and modifications without departing from the principles of the present disclosure, and such improvements and modifications should also be regarded as the protection scope of the present disclosure.

1. A water body pollution intelligent investigation method based on unmanned ships, comprising:
determining a first pollutant concentration value of a monitored water area according to water quality data of the monitored water area;
controlling an unmanned ship to cruise in the monitored water area according to a preset cruise trajectory and perform water quality collection to obtain a second pollutant concentration value of the monitored water area;
determining, when there is an abnormal cruise coordinate point, a target cruise trajectory according to the second pollutant concentration value of the abnormal cruise coordinate point, wherein a difference between the first pollutant concentration value and the second pollutant concentration value of the abnormal cruise coordinate point is greater than a preset threshold; and
controlling the unmanned ship to cruise according to the target cruise trajectory to determine a pollution source of the monitored water area.

2. The method according to claim 1, wherein the determining the first pollutant concentration value of the monitored water area according to the water quality data of the monitored water area comprises:
acquiring the water quality data of the monitored water area, wherein the water quality data comprises satellite data of the monitored water area and a plurality of sensor data; and
inputting the satellite data and the plurality of sensor data into a water area pollution analysis model to obtain the first pollutant concentration value, wherein the water area pollution analysis model is previously trained according to the water quality data.

3. The method according to claim 1, wherein the determining, when there is an abnormal cruise coordinate point, the target cruise trajectory according to the second pollutant concentration value of the abnormal cruise coordinate point comprises:
acquiring the abnormal cruise coordinate point;
determining a first cruise trajectory according to a circle with a preset radius centered on the abnormal cruise coordinate point;
controlling the unmanned ship to cruise according to the first cruise trajectory and collect at least two pollutant concentration values of at least two first cruise coordinate points; and
determining the target cruise trajectory according to the at least two pollutant concentration values.

4. The method according to claim 3, wherein the acquiring the abnormal cruise coordinate point comprises:
acquiring pollutant concentration values of a plurality of first cruise coordinate points in the preset cruise trajectory; and
sorting the pollutant concentration values of the plurality of first cruise coordinate points and determining that the first cruise coordinate point with the largest pollutant concentration value is the abnormal cruise coordinate point.

5. The method according to claim 3, wherein the acquiring the abnormal cruise coordinate point comprises:
acquiring pollutant concentration values corresponding to a plurality of first cruise coordinate points in the preset cruise trajectory;
acquiring at least two reference coordinate points of which pollutant concentration values are greater than a preset pollutant concentration threshold among the plurality of first cruise coordinate points; and
determining that a center point of the at least two reference coordinate points is the abnormal cruise coordinate point.

6. The method according to claim 1, wherein the controlling the unmanned ship to cruise according to the target cruise trajectory to determine the pollution source of the monitored water area comprises:
acquiring pollutant concentration values of a plurality of second cruise coordinate points in the target cruise trajectory;
determining that the second cruise coordinate point with the largest pollutant concentration value is a pollution source coordinate point of the pollution source; and
controlling the unmanned ship to perform water quality collection and image collection at the pollution source coordinate point.

7. A water body pollution intelligent investigation device, comprising:
a first determining unit, configured to determine a first pollutant concentration value of a monitored water area according to water quality data of the monitored water area;
a first control unit, configured to control an unmanned ship to cruise in the monitored water area according to a preset cruise trajectory and perform water quality collection to obtain a second pollutant concentration value of the monitored water area;
a second determining unit, configured to determine, when there is an abnormal cruise coordinate point, a target cruise trajectory according to the second pollutant concentration value of the abnormal cruise coordinate point, wherein a difference between the first pollutant concentration value and the second pollutant concentration value of the abnormal cruise coordinate point is greater than a preset threshold; and
a second control unit, configured to control the unmanned ship to cruise according to the target cruise trajectory to determine a pollution source of the monitored water area.

8. The device according to claim 7, wherein the first determining unit comprises:
an acquisition module, configured to acquire the water quality data of the monitored water area, wherein the water quality data comprises satellite data of the monitored water area and a plurality of sensor data; and
a processing module, configured to input the satellite data and the plurality of sensor data into a water area pollution analysis model to obtain the first pollutant concentration value, wherein the water area pollution analysis model is previously trained according to the water quality data.

9. (canceled)

10. A non-transitory machine-readable storage medium including instructions that, when accessed by a processor, cause the processor to:
determine a first pollutant concentration value of a monitored water area according to water quality data of the monitored water area;
control an unmanned ship to cruise in the monitored water area according to a preset cruise trajectory and perform water quality collection to obtain a second pollutant concentration value of the monitored water area;
determine, when there is an abnormal cruise coordinate point, a target cruise trajectory according to the second pollutant concentration value of the abnormal cruise coordinate point, wherein a difference between the first pollutant concentration value and the second pollutant concentration value of the abnormal cruise coordinate point is greater than a preset threshold; and
control the unmanned ship to cruise according to the target cruise trajectory to determine a pollution source of the monitored water area.

11. The non-transitory machine-readable storage medium to claim 10, wherein to determine the first pollutant concentration value of the monitored water area according to the water quality data of the monitored water area, the processor is further to:
acquire the water quality data of the monitored water area, wherein the water quality data comprises satellite data of the monitored water area and a plurality of sensor data; and
input the satellite data and the plurality of sensor data into a water area pollution analysis model to obtain the first pollutant concentration value, wherein the water area pollution analysis model is previously trained according to the water quality data.

12. The non-transitory machine-readable storage medium of claim 10, wherein to determine, when there is an abnormal cruise coordinate point, the target cruise trajectory according to the second pollutant concentration value of the abnormal cruise coordinate point, the processor is further to:
acquire the abnormal cruise coordinate point;
determine a first cruise trajectory according to a circle with a preset radius centered on the abnormal cruise coordinate point;
control the unmanned ship to cruise according to the first cruise trajectory and collect at least two pollutant concentration values of at least two first cruise coordinate points; and
determine the target cruise trajectory according to the at least two pollutant concentration values.

13. The non-transitory machine-readable storage medium of claim 12, wherein, to acquire the abnormal cruise coordinate point, the processor is to:
acquire pollutant concentration values of a plurality of first cruise coordinate points in the preset cruise trajectory; and
sort the pollutant concentration values of the plurality of first cruise coordinate points and determining that the first cruise coordinate point with the largest pollutant concentration value is the abnormal cruise coordinate point.

14. The non-transitory machine-readable storage medium of claim 12, wherein, to acquire the abnormal cruise coordinate point, the processor is further to:
acquire pollutant concentration values corresponding to a plurality of first cruise coordinate points in the preset cruise trajectory;
acquire at least two reference coordinate points of which pollutant concentration values are greater than a preset pollutant concentration threshold among the plurality of first cruise coordinate points; and
determine that a center point of the at least two reference coordinate points is the abnormal cruise coordinate point.

15. The non-transitory machine-readable storage medium of claim 10, wherein, to control the unmanned ship to cruise according to the target cruise trajectory to determine the pollution source of the monitored water area, the processor is further to:
acquire pollutant concentration values of a plurality of second cruise coordinate points in the target cruise trajectory;
determine that the second cruise coordinate point with the largest pollutant concentration value is a pollution source coordinate point of the pollution source; and
control the unmanned ship to perform water quality collection and image collection at the pollution source coordinate point.

16. The device of claim 7, further comprising the unmanned ship.
